(19) United States
(12) Patent Application Publication
(10) Pub. No.: US 2009/0024001 A1
(43) Pub. Date: Jan. 22, 2009

(54) MANOMETRY PROBE AND DATA VISUALIZATION

(76) Inventors: Thomas R. Parks, Hermosa Beach, CA (US); Jae S. Son, Rancho Palos Verdes, CA (US)

Correspondence Address: WOLF GREENFIELD & SACKS, P.C., 600 ATLANTIC AVENUE, BOSTON, MA, 02210-2206, US

(21) Appl. No.: 11/596,837

(22) PCT Filed: May 13, 2005
(86) PCT No.: PCT/US05/16809

§ 371 Date: Jun. 12, 2008

Related U.S. Application Data
(60) Provisional application No. 60/571,793, filed on May 17, 2004.

(51) Int. Cl. A61B 5/00; A61M 29/00

(52) U.S. Cl. 600/300; 606/191

(57) ABSTRACT

A manometry probe and data visualization system. An anorectal probe may include sensors disposed on a probe structure along a circumferential dimension of the probe. A data visualization system may provide visual representations of pressure information detected along a circumferential dimension of a region within an organism.

### RELATED APPLICATIONS

This application claims benefit under 35 U.S.C. § 119(e) to U.S. Provisional Application Ser. No. 60/571,793, titled “HIGH RESOLUTION ANORECTAL MANOMETRY PROBE AND DATA VISUALIZATION” filed on May 17, 2004, which is hereby incorporated by reference in its entirety.

### GOVERNMENT RIGHTS

The U.S. Government may have rights to one or more aspects of the invention set forth herein.

### BACKGROUND OF INVENTION

Normal function of the anorectum is important for continence and satisfactory bowel habit. Gastrointestinal motility disorders are significant both in terms of prevalence and morbidity (e.g., suffering, functional impairment, and health care resource use). Consequently, careful investigation may be essential to evaluate anorectal function.

Manometry is the detection of pressure information. Anorectal manometry is the detection of pressure information in and around the anorectal region (i.e., of the human body). Anorectal manometry may be used to investigate anorectal function.

Two different technologies are available for anorectal manometry: solid-state manometry probes and water perfuse manometry probes.

Solid-state manometry probes use solid state sensors to detect pressures within a region of an organism. Most probes incorporate between three and six sensors spaced several centimeters apart. The sensors are affixed to one side of the probe (i.e., within a 180° radial angle from the center axis of the probe) which may be inserted into the region of the organism (e.g., the anorectum). Thus, pressures can only be sensed on one side of the probe. Such systems are limited to detecting pressure information from only one “side” of the region at any given time, and must be repositioned to detected pressure information from another side of the region.

Water perfuse manometry systems pass water through several lumens (e.g., six to eight lumens) for measuring pressures within a region of an organism (e.g., the anorectum). The lumens are incorporated into a tube which may be inserted into the region. Each lumen terminates at an opening of the tube, which may be at any position along the length of the tube. During pressure sensing, water passes through each lumen and the opening into the region. The amount of water flow through each lumen may be measured, and the amount of water flow may provide an indication of the pressure within the region at the termination of the lumen. Water perfuse systems typically are cumbersome and messy to use.

### SUMMARY OF INVENTION

In an embodiment, a probe for detecting pressures within an organism is provided. The probe has a lengthwise dimension and a circumferential dimension. The circumferential dimension has a center axis. The probe includes a perimeter structure defining a perimeter of the probe in the circumferential and lengthwise dimensions. The probe also includes a first plurality of sensors disposed at positions on the perimeter structure at a first position along the lengthwise dimension. The positions along the circumferential dimension of at least three of the first plurality of sensors subtend, with respect to the center axis, a first angle of greater than 180 degrees. The probe also includes a second plurality of sensors disposed at positions on the perimeter structure at a second position along the lengthwise dimension. The positions along the circumferential dimension of at least three of the second plurality of sensors subtend, with respect to the center axis, a second angle of greater than 180 degrees.

In an aspect of this embodiment, the first and second angles are both greater than 270 degrees.

In another aspect of this embodiment, at least one of the first and second angles is approximately 360 degrees.

In another aspect of this embodiment, the probe has a substantially cylindrical shape.

In another aspect of this embodiment, the probe further includes a third plurality of sensors disposed at positions on the perimeter structure at a third position of the lengthwise dimension. The positions along the circumferential dimension of at least three of the third plurality of sensors subtend, with respect to the center axis, a third angle of greater than 180 degrees.

In another aspect of this embodiment, the probe includes at least 10 sensors.

In another aspect of this embodiment, the probe includes at least 50 sensors.

In another aspect of this embodiment, the probe includes at least 100 sensors.

In another aspect of this embodiment, the probe includes at least 200 sensors.

In another aspect of this embodiment, the probe includes 256 sensors.

In another aspect of this embodiment, the probe includes an array of sensors disposed on the perimeter structure at a plurality of positions along the lengthwise and circumferential positions.

In another aspect of this embodiment, the probe of further includes wires connected to the plurality of sensors and a relief feature coupled to the wires to couple wire stresses to the relief feature.

In another aspect of this embodiment, the probe further includes a balloon to stimulate the organism to induce a response and means for inflating the balloon.

In another aspect of this embodiment, the probe further includes a balloon to stimulate the organism to induce a response. The probe also includes a hollow portion disposed within the perimeter structure along the lengthwise dimension and a tube coupled to the balloon and extending through the hollow portion.

In another aspect of this embodiment, the probe further includes a handle for a human hand to hold the probe.

In another aspect of this embodiment, the organism is a human body, and the handle allows for alignment of the probe with particular human body geometry.

In another aspect of this embodiment, the positions of the plurality of sensors define a three-dimensional region within the organism.

In another aspect of this embodiment, the three-dimensional region is a cylinder.

In another embodiment, a probe for detecting pressures within an organism is provided. The probe has a lengthwise dimension and a circumferential dimension. The circumferential dimension has a center axis. The probe includes a perimeter structure defining a perimeter of the probe in the circumferential and lengthwise dimensions. The probe also includes means for detecting pressure information for a first set of positions and a second set of positions, the first set of positions being along the circumferential dimension of the probe at a first lengthwise position. The first set of positions subtend, with respect to the center axis, an angle of greater than 180 degrees. The second set of positions are along the circumferential dimension of the probe at a second lengthwise position. The second set of positions subtend, with respect to the center axis, an angle of greater than 180 degrees. The first and second sets of positions each include at least three positions.

In yet another embodiment, a method of detecting pressure values within a region of an organism having a lengthwise dimension and a circumferential dimension is provided. The circumferential dimension has a center axis. The method includes an act of detecting pressure information from a first set of positions along the circumferential dimension of the probe. The first set of positions subtend, with respect to the center axis, an angle of greater than 180 degrees. The method also includes an act of detecting, concurrently to detecting pressure information from the first set of positions, pressure information from a second set of positions along the circumferential dimension of the probe. The second set of positions subtend, with respect to the center axis, an angle of greater than 180 degrees. The first and second sets of positions each include at least three positions.

In yet another aspect, a method of visually representing pressure information detected by a plurality of sensors disposed at positions within a region of an organism is provided. The region has a lengthwise dimension and a circumferential dimension. The circumferential dimension has a center axis. The method includes an act of receiving pressure data representing the pressure information. The method also includes an act of providing, at least partially based on the pressure data, a first visual representation of the pressure information illustrating at least a portion of the region. The portion spans at least two positions along the lengthwise dimension and subtends, at each of the at least two positions along the lengthwise dimension, an angle greater than 180 degrees with respect to the center axis.

In an aspect of this embodiment, the angle is greater than 270 degrees.

In another aspect of this embodiment, the angle is approximately 360 degrees.

In another aspect of this embodiment, the a polar plot illustrating a circumferential pressure distribution representing pressure values detected within the portion of the region is provided.

In another aspect of this embodiment, the polar plot includes a line trace.

In another aspect of this embodiment, the polar plot includes a tone-encoded plot.

In another aspect of this embodiment, the first visual representation is displayed on a user interface display, and the method further includes providing a user-movable control on the user interface display. The control enables a user to select a position along the lengthwise dimension of the probe for which the first visual representation is provided.

In another aspect of this embodiment, the first visual representation is a multi-dimensional representation representing the pressure information detected at a plurality of positions along the lengthwise dimension and circumferential dimension of the region.

In another aspect of this embodiment, the first visual representation is a two-dimensional plot representing the pressure information at a plurality of positions along the lengthwise dimension and circumferential dimension of the region.

In another aspect of this embodiment, the first visual representation is a contour plot.

In another aspect of this embodiment, the first visual representation includes a three-dimensional mesh plot including a mesh.

In another aspect of this embodiment, the pressure information is represented at least partially by a radial location of at least a portion of the mesh. The radial location is along a radial dimension of the mesh plot. The radial dimension represents a dimension of the region of the organism that is substantially orthogonal to the lengthwise dimension.

In another aspect of this embodiment, the pressure information is represented at least partially by a tone-encoding of the three-dimensional mesh plot.

In another aspect of this embodiment, a second visual representation representing the pressure information detected at a plurality of positions along the lengthwise dimension of the region is provided. Each position of the plurality of positions having substantially a same circumferential position along the circumferential dimension.

In another aspect of this embodiment, the first and second visual representations are displayed concurrently.

In another aspect of this embodiment, the second visual representation includes a line trace.

In another aspect of this embodiment, the second visual representation includes a tone-encoded plot.

In another aspect of this embodiment, the first visual representation is displayed on a user interface display, and a user-movable control is provided on the user interface display. The control enables a user to select a circumferential position along the circumferential dimension of the probe for which the second visual representation is to be provided.

In another aspect of this embodiment, the pressure information was detected by the plurality of sensors disposed at the positions within the region over a period of time such that the pressure information represents the pressure information detected over the period of time. The second visual representation is altered to represent a change in the pressure information over the period of time.

In another aspect of this embodiment, the second visual representation represents an average pressure value, maximum pressure value, and/or minimum pressure value of pressure values detected during a particular time period.

In another aspect of this embodiment, the second visual representation represents, for a plurality of lengthwise positions along the lengthwise dimension of the region, average pressure values, maximum pressure values and/or minimum pressure values determined from pressure values detected at a plurality of positions along the circumferential dimension of the region at the each of the plurality of lengthwise positions.

In another aspect of this embodiment updated pressure data representing the pressure information is received. A second visual representation is provided at least partially based on the updated pressure data, illustrating at least a portion of the region. The portion subtends, with respect to the center axis, an angle greater than 180 degrees.

In another aspect of this embodiment, the first visual representation is displayed on a human perceptible medium and the second visual representation is displayed on the human perceptible medium. The second visual representation is displayed soon enough in time after the first visual representation such that the first and second visual representations appear on the human perceptible medium as a single, moving temporal representation.

In another aspect of this embodiment, the first visual representation represents an average pressure value, maximum pressure value, and/or minimum pressure value of pressure values detected during a particular time period.

In another aspect of this embodiment, the first visual representation represents, for a plurality of circumferential positions along the circumferential dimension of the region, average pressure values, maximum pressure values and/or minimum pressure values determined from pressure values detected at a plurality of positions along the lengthwise dimension of the region at the each of the plurality of circumferential positions.

In another aspect of this embodiment, interpolated pressure values at least partially based on the pressure information are provided. The interpolated pressure values represent pressure information for positions between the positions of the plurality of sensors.

In yet another embodiment, the invention is directed to a computer program product. The computer program product includes a computer-readable medium. The computer program product also includes computer-readable signals, stored on the computer-readable medium, that define instructions that, as a result of being executed by a computer, control the computer to perform a method of visually representing pressure information detected by a plurality of sensors disposed at positions within a region of an organism. The region has a lengthwise dimension and a circumferential dimension. The circumferential dimension has a center axis. The method includes an act of receiving pressure data representing the pressure information. The method also includes an act of providing, at least partially based on the pressure data, a first visual representation of the pressure information illustrating at least a portion of the region. The portion spans at least two positions along the lengthwise dimension and subtends, at each of the at least two positions along the lengthwise dimension, an angle greater than 180 degrees with respect to the center axis.

In a further embodiment, the invention is directed to a system for visually representing pressure information detected by a plurality of sensors disposed at positions within a region of an organism. The region has a lengthwise dimension and a circumferential dimension. The circumferential dimension has a center axis the system includes a visualization component operative to receive pressure data representing the pressure information and provide, at least partially based on the pressure data, a first visual representation of the pressure information illustrating at least a portion of the region. The portion spans at least two positions along the lengthwise dimension and subtends, at each of the at least two positions along the lengthwise dimension, an angle greater than 180 degrees with respect to the center axis.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a block diagram illustrating an example of a system for providing representations of pressure data to a user, according to some embodiments of the invention;

FIG. 2A is a diagram illustrating an example of a detection component for detecting manometry data, according to some embodiments of the invention;

FIG. 2B is a diagram illustrating an example of a portion of a circumferential section of a probe, according to some embodiments of the invention;

FIG. 2C is a diagram illustrating an example of an end-view of a probe, according to some embodiments of the invention;

FIG. 3 is a screenshot illustrating an example of a user interface display including a three-dimensional mesh plot;

FIG. 4 is a screenshot illustrating an example of a user interface display including three-dimensional mesh plots representing pressure information in different orientations;

FIG. 5 is a flowchart illustrating an example of a method of visually representing pressure information, according to some embodiments of the invention;

FIG. 6 is a screenshot illustrating an example of a user interface display, including multiple visual representations of pressure information;

FIG. 7 is a screenshot illustrating an example of a user interface display, including multiple visual representations of pressure information;

FIG. 8 is a screenshot illustrating an example of a user interface display, including a temporal representation of pressure information;

FIG. 9 is a block diagram illustrating an example of a computer system; and

FIG. 10 is a diagram illustrating an example of data transfer.

### DETAILED DESCRIPTION

A manometry probe having sensors disposed around the circumferential dimension of the probe (e.g., as much as 180° or greater, even as much as 270°, or even greater, up to approximately 360°) may provide pressure data representing pressure information detected within a region (e.g., the anorectum) of an organism (e.g., the human body). By detecting pressures around the circumferential dimension of the probe, a more complete picture of the pressures exerted within the region may be obtained than previously obtainable using known manometry probes, which can only detect data from one side of the region at a time. Such a probe and the data it provides may be used to implement improved manometry visualization and analysis techniques, as described below. Visualization of pressure information detected around the circumferential dimension of a region (e.g., the anorectum) may facilitate analysis of the region (by a physician or other persons) and the diagnoses of disorders.

Although aspects of the invention described below are described primarily in relation to visually indicating values of physical properties (e.g., pressure, pH level, temperature, voltage, tissue impedance) detected from an organism (e.g., a human), or values derived therefrom, over time, such aspects are not limited thereto, but may be applied to visually indicating any type of values over time. Further, although aspects of the invention described below are described primarily in relation to visually indicating values of physical properties detected within the anorectum, such aspects are not limited thereto, but apply to visually indicating physical properties detected within other organs or combinations of organs, including tubular organs, located within an organism such as, for example, the esophagus, duodenum, small bowel, bile duct, colon, urethra and Sphincter of Oddi. Further, such values may be detected along a spatial dimension external to an organism, for example, on an exterior surface of an organism.

Embodiments of the invention may employ systems and methods, including visualization techniques, described in co-pending U.S. patent application titled “ANALYSIS AND VISUALIZATION METHODS USING MANOMETRY DATA” by Tom Parks, filed on even date herewith under attorney docket No. S1489.70004US01, which is hereby incorporated by reference in its entirety.

The function and advantage of these and other embodiments of the present invention will be more fully understood from the examples described below. The following examples are intended to facilitate an understanding of aspects of the present invention and their benefits, but do not exemplify the full scope of the invention.

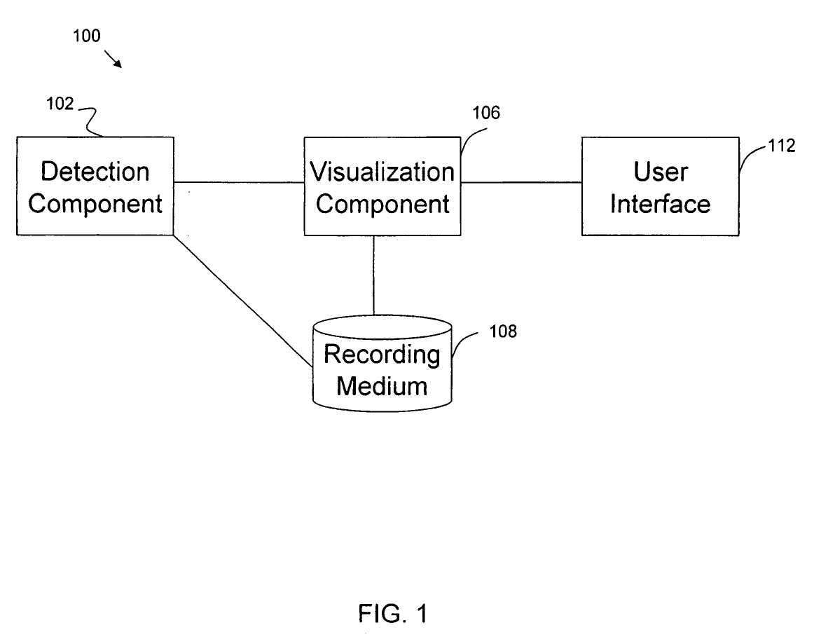

FIG. 1 is a block diagram illustrating an example of a system 100 for detecting values (e.g., pressure values) within an organism and visually indicating the detected values to a user. System 100 may include any of a detection component 102, a visualization component 106, a recording medium 108, a user interface 112, other components or any suitable combination of the foregoing.

As used herein, a “user interface” is an application or part of an application (i.e., a set of computer-readable instructions) that enables a user to interface with an application during execution of the application. A user interface may include code defining how an application outputs information to a user during execution of the application, for example, visually through a computer screen or other means, audibly through a speaker of other means, and manually through a game controller or other means. Such user interface also may include code defining how a user may input information during execution of the application, for example, audibly using a microphone or manually using a keyboard, mouse, game controller, track ball, touch screen or other means.

The user interface 112 may define how information is visually presented (i.e., displayed) to the user, and defines how the user can navigate the visual presentation (i.e., display) of information and input information in the context of the visual presentation. During execution of the application, the user interface may control the visual presentation of information and enable the user to navigate the visual presentation and enter information in the context of the visual presentation. Types of user interfaces range from command-driven interfaces, where users type commands, menu-driven interfaces, where users select information from menus, and combinations thereof, to GUIs, which typically take more advantage of a computer's graphics capabilities, are more flexible, intuitive and easy to navigate and have a more appealing “look-and-feel” than command-driven and menu-driven visual user interfaces. As used herein, the visual presentation of information presented by a user interface or GUI is referred to as a “user interface display” or a “GUI display”, respectively.

The detection component 102 may detect pressure information of an organism (e.g., from within an organism) over a period of time. Detection component 102 may be configured to provide pressure data, including pressure values representing the pressure information, to a visualization component 106 and/or a recording medium 108. For example, as will be described in more detail below, if the pressure information is to be visually indicated in real time, then the pressure information is provided to at least the visualization component 106 and also may be persisted in a recording medium 108. If the pressure information is not to be visually indicated in real time, but is to be visually indicated post hoc at a later point in time, then the detection component 102 may provide the pressure data to the recording medium 108 but not to the visualization component 106.

Visualization component 106 may be operable to receive pressure data from detection component 102 (e.g., for real time visual indication) and from recording medium 108 (e.g., for post hoc visual indication). Further, the visualization component may be operable to send data to be persisted to the recording medium during or after visually indicating information to a user. Such information may include the pressure values themselves, display information such as values for display parameters, locations of anatomical landmarks, locations of a probe (e.g., catheter) with respect to an organism, interpolated values, etc. The visualization component 106 also may be operable to receive user input from user interface 112, which may be originated from any of a variety of user input devices (e.g., any of those described above). The visualization component 106 may include any of a variety of logic for generating data to send to the user interface 112 based on the pressure data and received user input.

Visualization component 106 may be configured to perform operations on the pressure data, provide for displaying representation(s) of the pressure information and/or perform other operations. Visualization component 106 may be configured to perform one or more acts of method 700 and other methods described in further detail below.

System 100 and components thereof (e.g., visualization component 106) may be implemented using any of a variety of technologies, including software (e.g., C, C#, C++, Java, or a combination thereof), hardware (e.g., one or more application-specific integrated circuits), firmware (e.g., electrically-programmed memory) or any combination thereof. One or more of the components of system 100 may reside on a single device (e.g., a computer), or one or more components may reside on separate, discrete devices. Further, each component may be distributed across multiple devices, and one or more of the devices may be interconnected.

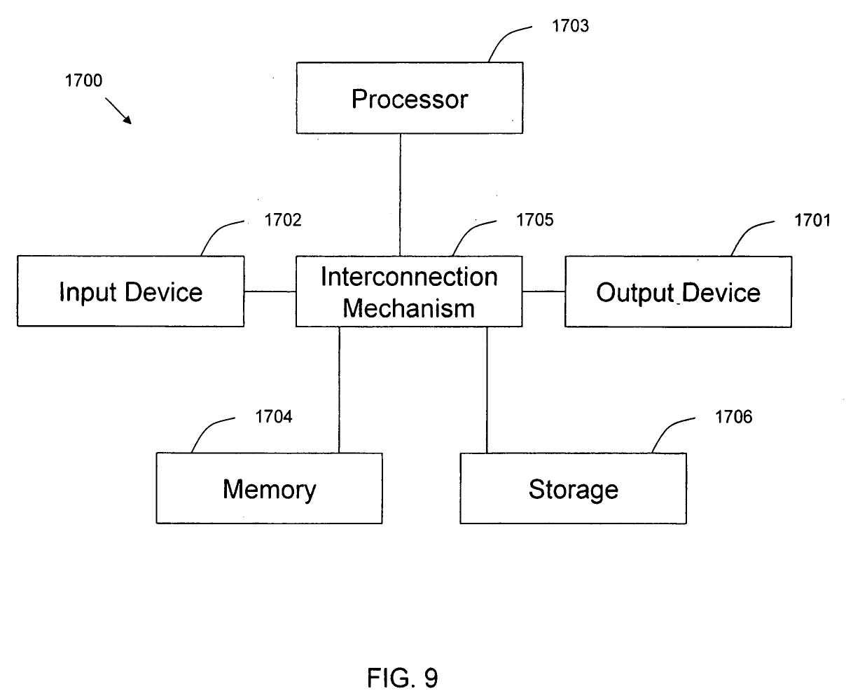

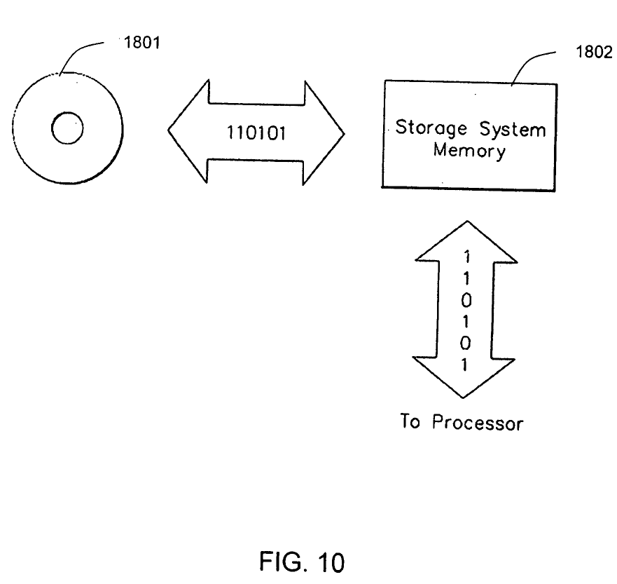

Further, on each of the one or more devices that include one or more components of system 100, each of the components may reside in one or more locations on the system. For example, different portions of the components of these systems may reside in different areas of memory (e.g., RAM, ROM, disk, etc.) on the device. Each of such one or more devices may include, among other components, a plurality of known components such as one or more processors, a memory system, a disk storage system, one or more network interfaces, and one or more busses or other internal communication links interconnecting the various components. System 100, and components thereof, may be implemented using a computer system such as that described below in relation to FIGS. 9 and 10.

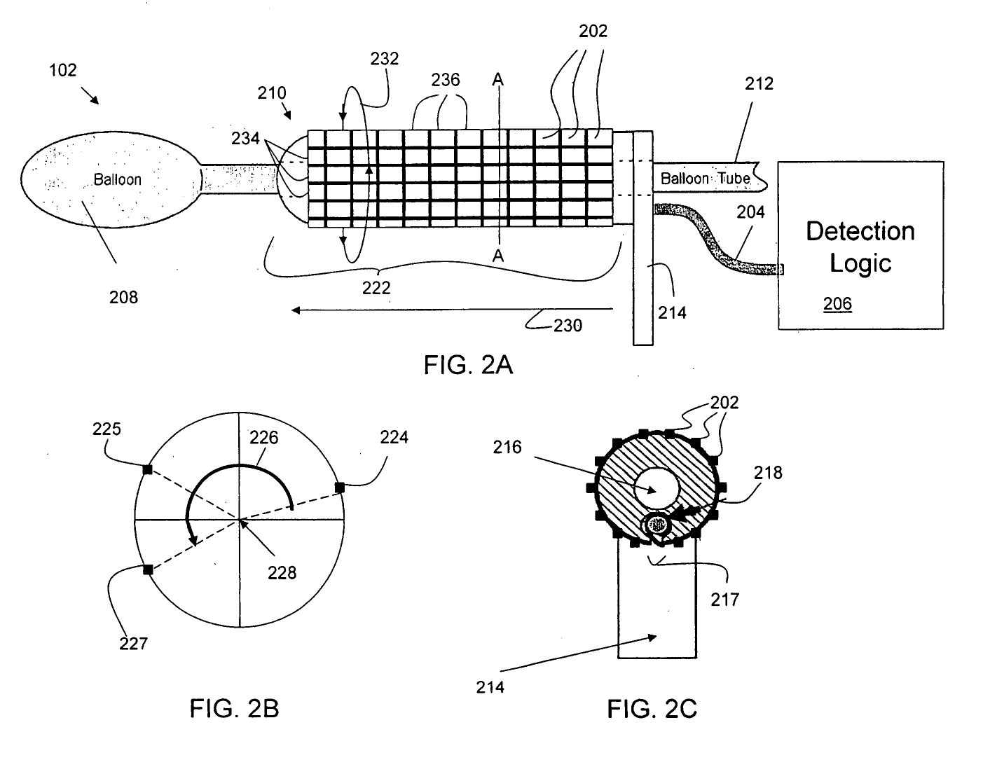

FIG. 2A is a sketch illustrating an example of detection component 102, according to some embodiments of the invention. Detection component 102 may include a probe 210 which may be used for detecting pressure information within an organism, e.g., within the anorectum of the human body. Probe 210 may include a perimeter structure 222 which may define a perimeter of probe 210 along a lengthwise dimension and a circumferential dimension. Perimeter structure 222 may be formed of any suitable material, e.g., plastic or metal.

Although probe 210 is illustrated in FIG. 2A as having a substantially circular cross-section, the invention is not so limited. The cross-section of probe 210 may have any of a variety of shapes such as, for example: elliptical, rectangular (e.g., a square), triangular, an irregular shape, another shape or any suitable combination of the foregoing.

Probe 210 may include a plurality of sensors 202 disposed at positions on perimeter structure 222. Any suitable type of sensors may be used. In one embodiment, sensors 202 may be capacitive sensors. Capacitive sensors may be formed by forming a lengthwise set of electrodes 234 (e.g., metal traces) along a lengthwise dimension 230 at multiple circumferential positions along a circumferential dimension 232 of probe 210. A circumferential set of electrodes 236 may be formed along the circumferential dimension 232 at multiple lengthwise positions along the lengthwise dimension 230 of probe 210. A compliant dielectric layer (not shown) may be formed between the lengthwise set of electrodes and the circumferential set of electrodes. As the pressure at a position of probe 210 increases, the distance between a lengthwise electrode and a circumferential electrode at that position may decrease. A decrease in distance between the lengthwise electrode and the circumferential electrode at the position may cause a capacitance increase between the electrodes. By measuring the capacitance between the lengthwise electrode and the circumferential electrode at the position, an indication of the pressure at the position may be obtained. A sensor 202 may be a region of overlap of a lengthwise electrode and a circumferential electrode.

Sensors 202 may be disposed on the perimeter structure in any suitable configuration. For example, in FIG. 2A sensors 202 are configured in an array along the lengthwise dimension 230 and the circumferential dimension 232 of the probe 210. Any suitable number of sensors 202 may be disposed on perimeter structure 222, e.g., greater than 10, greater than 50, greater than 100 and/or greater than 200 sensors. In some embodiments, sensors 202 may be configured in an array that includes two hundred fifty-six sensors. The array may include sensors positioned at sixteen different lengthwise positions on perimeter structure 222. For example, at each of these sixteen different lengthwise positions, sixteen sensors may be positioned at sixteen respective circumferential positions. It should be appreciated that sensors may be positioned at any number of lengthwise positions (e.g., greater or less than 16) and/or any number of circumferential positions (e.g., greater than or less than 16).

Each of sensors 202 may be configured to sense any of a variety of physical properties, for example, pressure, pH, temperature, voltage, tissue impedance, another physical property or any combination thereof.

FIG. 2B is a sketch illustrating a simplified cross-sectional view of a circumferential section of probe 210 at a position along the lengthwise dimension 230 indicated by A-A. As discussed above, sensors 202 may be positioned along the circumferential dimension of probe 210. FIG. 2B illustrates three sensors 202 at three respective circumferential positions 224, 225, and 227. Circumferential positions 224, 225, and 227 subtend an angle 226 with respect to the center axis 228. Providing sensors at circumferential positions that subtend an angle of greater than 180° (up to approximately 360°) may enable obtaining more complete pressure information about a region of an organism than previously obtainable using known probes.

As used herein, the “center axis” of the circumferential dimension is an axis of symmetry of the circumferential perimeter of the probe and/or a region for which pressure is detected along the length of the probe and/or the region. If the probe and/or the region has a uniform cross-sectional shape (e.g., circle, ellipse, rectangle (e.g., square)) along its length, then the center axis is a straight line, the line passing through the center of symmetry at each position along the lengthwise dimension. The center of symmetry, at each lengthwise position along the length of the probe and/or the region, is a point which serves as the mid-point for any line drawn through the point from one point on the perimeter of the probe and/or the region to another point on the perimeter of the probe and/or the region. It should be appreciated that there will always be irregularities, however minimal, in the shape of the probe (particularly if pressure is being exerted on the probe) and/or the region such that the center axis is never a perfectly straight line, and the center of symmetry for a particular lengthwise position may not be an exact center of symmetry. Further, in some embodiments of the invention, the probe and/or the region may have a non-uniform cross-section along its length. Thus, the center axis and the center of symmetry at a lengthwise position are typically, to some extent, an approximation.

Sensors 224, 225, and 227 need not necessarily lie in the same plane, and need not necessarily have the same position along the lengthwise dimension 230 of probe 210. For example, sensor 224 may have a different position along the lengthwise dimension of probe 210 than sensors 225 and 227. To determine the angle that the circumferential positions of sensors 224, 225, and 227 subtend, one may consider a simplified cross-sectional view (as illustrated in FIG. 2B) in which the positions of sensors 224, 225, and 227 are represented as being in the same plane, even though sensors 224, 225, and 227 may not actually be in the same plane, as each may have a different lengthwise position. Further, the cross-section of probe 210 need not necessarily be uniform along the entire lengthwise dimension. For example, probe 210 may be tapered at one end.

In some embodiments of the invention, angle 226 may be greater than 180°, as much as 270° or even greater. For example, angle 226 may be approximately 360°.

FIG. 2C is a sketch illustrating an example of a back-end view of probe 210; that is, from a viewpoint in back of probe 210 (e.g., from the right side of FIG. 2A looking toward the left side). Sensors 202 may be disposed at several respective positions along the circumferential dimension of probe 210. In some embodiments, sensors 202 are located at positions encompassing almost the entire circumference of probe 210 (i.e., approximately 360°).

Probe 210 may include a hollow tube 216 that extends through perimeter structure 222, and a balloon tube 212 may extend through hollow tube 216. Balloon tube 212 may be any suitable tube through which gas may flow for inflating a balloon 208, may be inflated by air (or another gas) passing through balloon tube 212. Balloon 208 may be any suitable type of balloon, and may be formed of any suitable material, e.g., latex, rubber, or plastic. Balloon 208 may be used to induce a response in a region of an organism in which the probe 210 is positioned.

Probe 210 also may include a handle 214, which may be affixed (e.g., mechanically or otherwise) to perimeter structure 222. Handle 214 may be used by a user of probe 210 (e.g., a physician) for holding probe 210 while inserting probe 210 into a region of an organism and/or aligning probe 210 with respect to the particular geometry of the organism. For example, in some embodiments in which the probe 210 is used for anorectal analysis, handle 214 may be constructed and arranged to wedge between the buttocks of a human body, so that probe 210 is aligned in a particular orientation with respect to the anorectal region. Orienting probe 210 as such may provide a point of reference for the positions of the detected pressure information. Handle 214 may extend outward radially from the central axis as illustrated in FIG. 2A, may extend outward in the lengthwise dimension of probe 210, or some combination thereof.

Probe 210 may include a relief feature 218. Relief feature 218 may provide for strain relief of transmission media 204. For example, if transmission media 204 are wires connected to sensors 202, relief feature 218 may be a mechanical component (e.g., a post attached to perimeter structure 222) around which the wires may be wound. By winding the wires around the relief feature 218, mechanical stresses that may be applied to the wires (e.g., incidentally) may be coupled to the relief feature 218, rather that to sensors. Such stress relief may be desirable to preserve and protect sensors 202 and their electrical interconnections.

As illustrated in FIG. 2A, detection component 102 also may include a transmission medium 204, which may be used for transmitting signals from sensors 202 to detection logic 206. The transmission medium 204 may be any of a plurality of types of transmission media, such as a group of wires (e.g., a bus), a wire, a cable, an optical fiber, a group of optical fibers or a wireless transmission medium (e.g., carrier waves through air). The transmission medium 204 may carry control and addressing signals from the detection logic to the sensors 202 and may carry detected values from the sensors 202 to the detection logic 206.

Detection logic 206 may be configured to receive detected values from sensors 202 via the transmission medium 204, and may perform processing on detected values. The detection logic 206 may include signal processing logic to process the signals carrying the values received over transmission medium 204. For example, the signal process logic may include noise filtering logic, analog-to-digital conversion logic and other logic to convert the raw detected values into a suitable form to be input to visualization component 106.

Probe 210 may be inserted into a human or another organism, for example, the anorectum of such an organism. Probe 210 may be constructed with a length long enough to span both the internal and external anal sphincters. For example, probe 210 may span approximately five centimeters (cm) in the lengthwise direction. Probe 210 may have a diameter through the central axis that may span approximately half of a centimeter to three centimeters. It should be appreciated that the invention is not limited to a probe having a length of five cm, as the probe may have other lengths intended to fall within the scope of the invention. For example, the probe may be constructed and arranged to have a length suitable for use in a particular region of an organism.

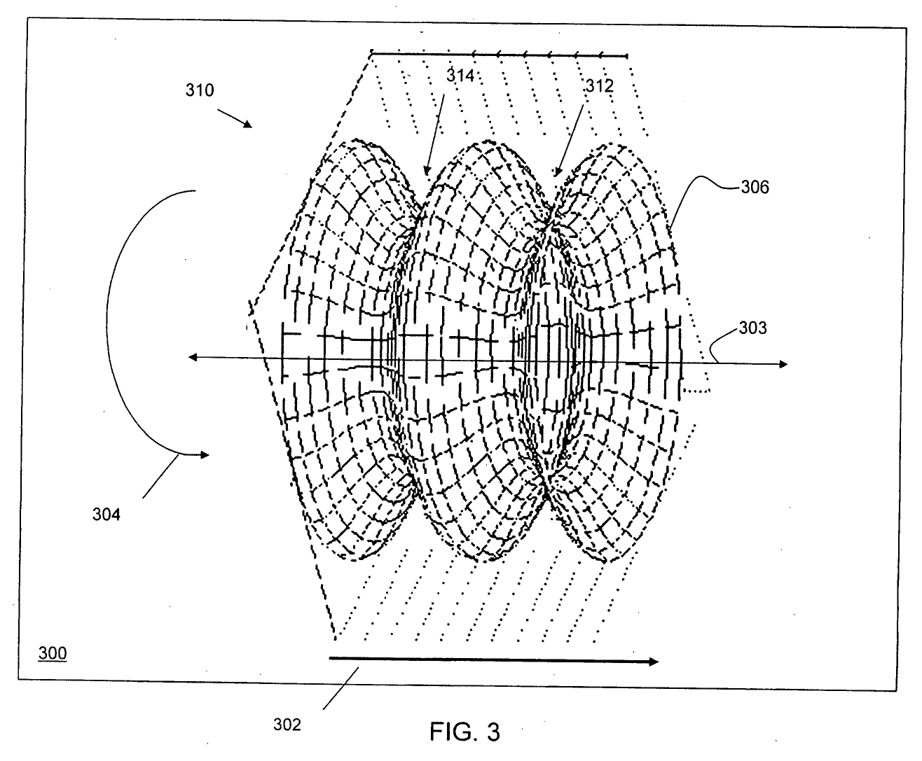

FIG. 3 is a screen shot illustrating an example of a user interface display 300, which may be provided by user interface 112, according to some embodiments of the invention. In this example, display 300 includes an example of a sub-display 310 for providing visual representations of pressure data to a user. Display 300 may include one or more sub-displays. A sub-display may provide any of a variety of types of visual representations to a user, e.g., a multi-dimensional plot, a contour plot, a line trace plot and/or a polar plot, for example, as described below in more detail.

In this example, sub-display 310 displays a three-dimensional mesh plot 306. Three dimensional mesh plot 306 may represent to a user pressure information detected by sensors positioned along the lengthwise dimension 302 and the circumferential dimension 304 of probe 210 (e.g., positioned in any of the configurations described above).

Pressure values detected at respective positions along the lengthwise and circumferential dimensions 302 and 304 of probe 210 may be represented by three-dimensional mesh plot 306 in various ways. In the example illustrated in FIG. 3, each location along the lengthwise and circumferential dimensions 302 and 304 within sub-display 310 corresponds to the position on probe 210 at which the pressure represented by the pressure value was detected. At each location, the pressure value may be represented as the mesh's distance from the center axis 303 of circumferential dimension 304. In some embodiments, a relatively high pressure value may be represented, at the corresponding location, by a relatively small distance of the mesh from the center axis, and a relatively low pressure value may be represented, at the corresponding location, by a relatively large distance of the mesh from the center axis. Alternatively, a relatively high pressure value may be represented, at the corresponding location, by a relatively large distance of the mesh from the center axis, and a relatively low pressure value may be represented, at the corresponding location, by a relatively small distance of the mesh from the center axis.

By viewing a visual representation of pressure data, e.g., three-dimensional mesh plot 306, a user (e.g., a physician) may obtain information about pressures within a region of an organism, e.g., the anorectum. Further, a user may identify one or more aspects of the region by viewing visual representations of pressure data. Obtaining information about pressures in the region and identifying one or more aspects of the region may facilitate determining characteristics of the region, e.g., assessing anomalies and diagnosing disorders.

For example, the anorectum may have an internal anal sphincter and an external anal sphincter, which are muscles that are important to bowel function. A physician may view a visual representation of pressure information within the anorectum (e.g., contour plot 306), identify the location of one or more aspects (e.g., sphincters) within the visual representation and determine characteristics of the anorectum.

A portion of a region in the vicinity of a sphincter may have higher pressure values than other portions of the region. One may identify a location along the lengthwise dimension 302 of sub-display 310 that corresponds to a sphincter position by observing the distance of the mesh from the center axis. For example, the location 312 along the lengthwise dimension 302 within sub-display 310 may correspond to a position of an external anal sphincter. The location 314 along the lengthwise dimension 302 may correspond to the position of the internal anal sphincter within the anorectum.

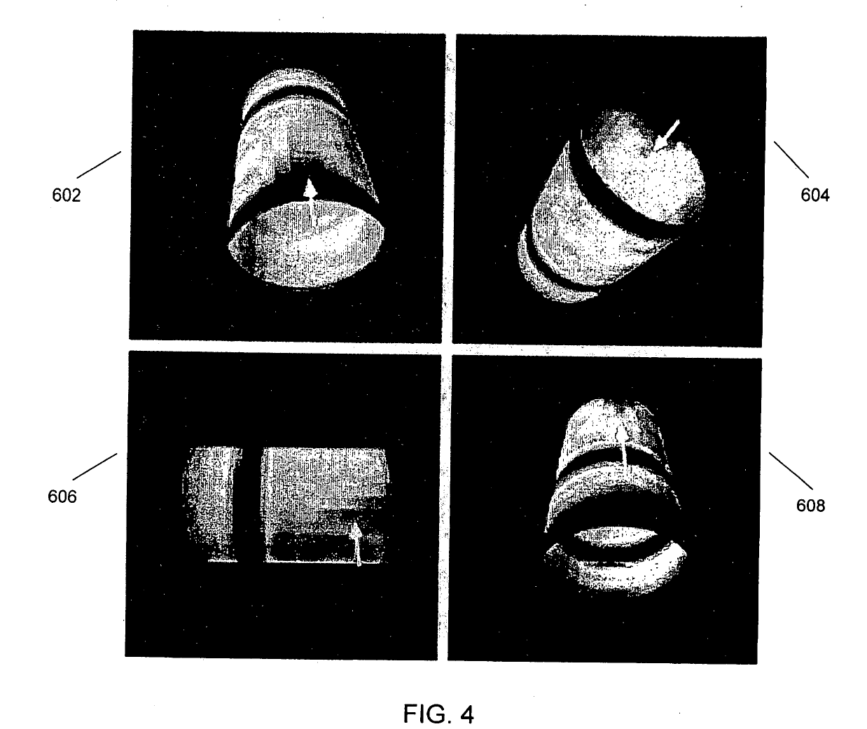

FIG. 4 is a screen shot illustrating four examples of sub-displays 602, 604, 606, and 608, according to some embodiments of the invention. In these embodiments, sub-displays 602, 604, 606, and 608 each include a three-dimensional mesh plot of substantially cylindrical shape.

In some embodiments of these sub-displays, pressure values may be represented by multiple tones corresponding to multiple ranges of pressure values that correspond to the pressure data. Each tone may represent a respective range of pressure values. The size (i.e., granularity) of the ranges may be uniform. The uniform granularity of the ranges of pressure values may vary, and may be so fine that the spectrum of tones seems continuous to the human eye. A tone may be any suitable visually differentiating characteristic. For example, each tone may be any of a variety of colors or shades of gray, or any other suitable visually differentiating characteristic. Display 300 may include a tone bar (not shown) which may be a legend which maps a tone to a corresponding pressure range. For example, in some embodiments, the color blue may represent a low pressure range and the color red may represent a high pressure range.

In some aspects of the invention, two or more tone ranges may represent value ranges of different size; i.e., the tone ranges may be of non-uniform granularity. A control may be provided (e.g., by user interface 112) that enables a user to set and modify such different ranges. For example, the user may define one tone range to represent a relatively small range of pressure values, thereby providing a relatively high visual resolution in a pressure range of interest. Another tone may be defined to represent a relatively wide pressure interval thereby providing a relatively low visual resolution.

Lengthwise positions of pressure-producing anatomical elements such as sphincters may be determined using a color representation (or a tone representation) similar to the visual representations described with respect to FIGS. 3 and 4.

In some embodiments, pressure values may be represented in multiple different ways simultaneously, e.g., by multiple tones and by the distance of a mesh from a center axis of the circumferential dimension.

In some embodiments, a visual representation may be movable by a user to view the visual representation from another perspective. FIG. 4 illustrates four examples of sub-displays 602, 604, 606, and 608, which each may represent the same pressure data from a different perspective. A control may be provided for a user to change the perspective shown in a sub-display. In one embodiment, a user may locate a cursor on a sub-display (e.g., 602) and depress a button (e.g., a mouse button), and then move the cursor while keeping the button depressed. As the cursor is moved, sub-display 602 may rotate to show a different perspective based on the direction in which the cursor is moved and may maintain the different perspective when the user releases the button, for example, any of the perspectives illustrated in sub-displays 604, 606, or 608.

A sub-display (e.g., any of sub-displays 310, 602, 604, 606, and 608 and/or any other suitable sub-display discussed below) may include a visual representation of pressure values at circumferential locations subtending an angle of approximately 360°. In some embodiments, some of the pressure values are interpolated from actually detected values. For example, the pressure sensors may be positioned, and detect pressure from, almost 360° around a circumferential dimension of a region. For example, referring to FIG. 2C, sensors 202 may be positioned all around a perimeter of probe 210, except at perimeter area 217. Pressure values for the missing degrees of the circumferential dimension may be interpolated from values detected at other positions, including positions adjacent to the missing degrees. Thus, 360° of pressure data may be represented although slightly less than 360° of pressure information may have been detected. Pressure values may be interpolated in any suitable spatial dimension. In some embodiments, the pressure data may be made quasi-continuous even though pressures may be detected at discrete times and/or positions. Pressure data may be made quasi-continuous by including interpolated pressure values in the pressure data. A quasi-continuous visual representation (e.g., having smooth changes) may be provided based on quasi-continuous pressure data. Any visual representation discussed herein may be quasi-continuous.

In some embodiments, a two-dimensional plot of pressure data may be provided that represents pressure information detected along a lengthwise and circumferential dimension of a region. In some types of two-dimensional plots, one dimension of the plot may represent the circumferential dimension of the region and one dimension may represent the lengthwise dimension of the region. For example, a horizontal dimension may represent the circumferential dimension and a vertical dimension may represent the lengthwise dimension, or vice-versa. Providing a two dimensional plot may enable concurrent visualization of substantially an entire portion of a region in which pressure information is detected, which may not be achievable using a three-dimensional mesh (e.g., three-dimensional mesh plot 306 of FIG. 3).

By way of illustration, a two-dimensional plot may be similar to the visual representation illustrated in sub-display 602, however the cylinder illustrated in sub-display 602 may be “unwrapped” such that it lies in a plane. A two-dimensional plot may be a tone plot, a contour plot, a line trace plot (e.g., having several line traces), or any other suitable type of two-dimensional plot.

Providing a visual representation of pressure values detected within at least a portion of a region within an organism may facilitate diagnoses and/or assessment of the function of the region. It may be desirable to provide a visual representation of pressure information detected at least 180° (up to approximately 360°) around a circumference of a region or a portion thereof, i.e., illustrating a portion of a region subtended by at least a 180° angle with respect to the center axis of a circumference of the region.

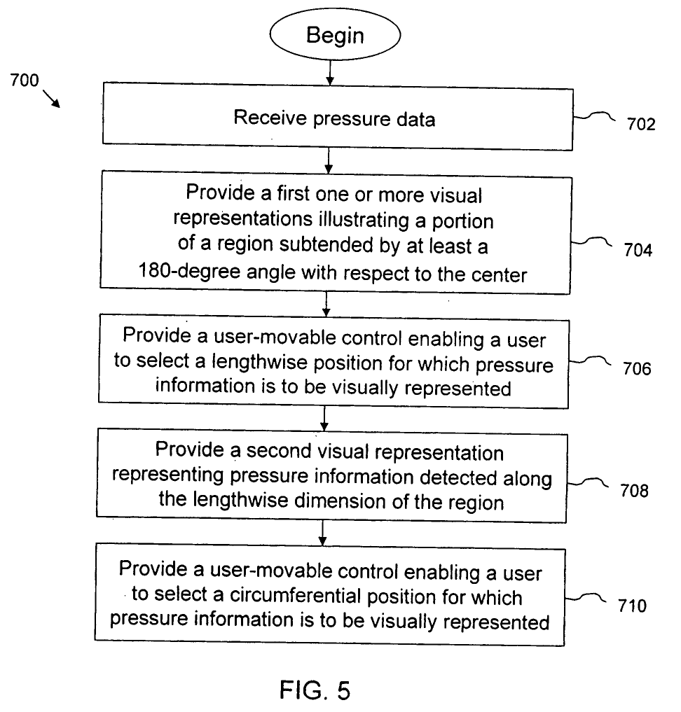

FIG. 5 is a flowchart illustrating an example of a method 700 of providing a visual representation of pressure data, according to some embodiments of the invention. Method 700 may be implemented at least partially using visualization component 106.

In act 702, pressure data may be received which represents pressure information detected within a region of an organism. Pressure data may be received from detection component 102, and may be received from a plurality of sensors disposed at positions within a region of an organism. The region may have a lengthwise dimension and a circumferential dimension having a center axis. At least three of the sensors may have positions along the circumferential dimension that subtend, with respect to the center axis of the circumferential dimension, an angle of at least 180°, possibly up to approximately 360°. The number and configuration (including positions) of the sensors that detected the pressure information may be any of a variety of numbers and configurations, e.g., any of those described above in relation to FIGS. 2A-2C. Act 702 may be performed at least partially by visualization component 106.

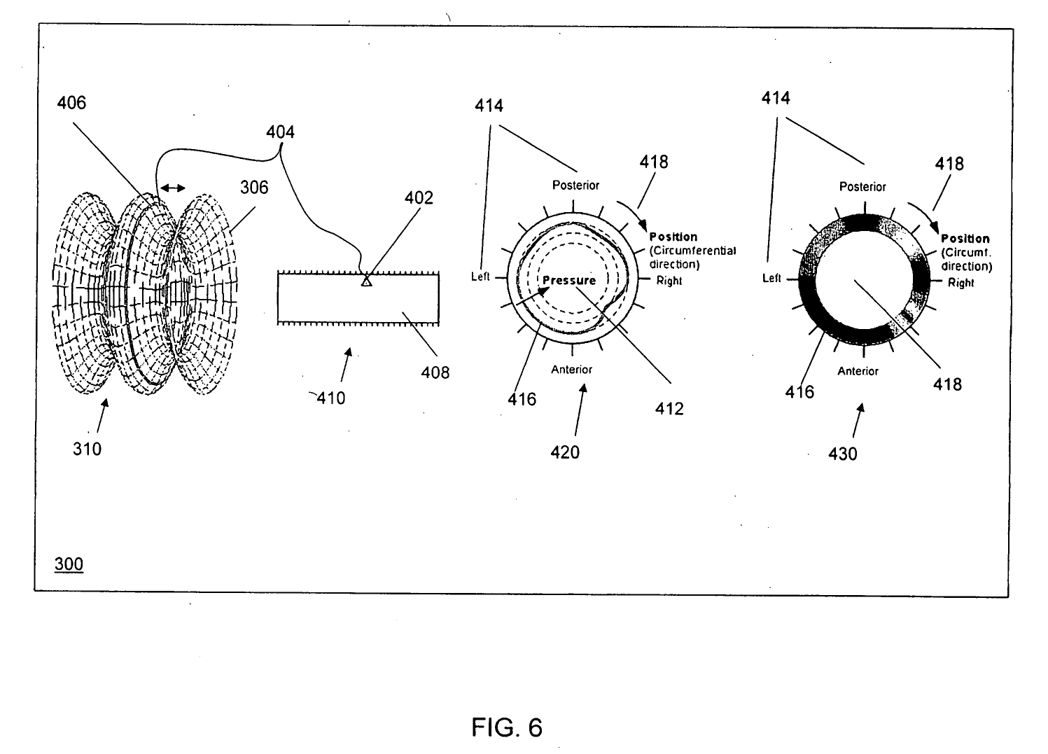

In act 704, a visual representation may be provided illustrating a portion of a region, the portion subtending at least a 180° angle with respect to the center axis, possibly up to approximately 360°. In some embodiments, more than one visual representation may be provided, as will be described in more detail below. One or more visual representations illustrated in FIG. 6 may be provided in act 704, e.g., three-dimensional mesh plot 306, polar line trace plot 412 and polar tone plot 418. Act 704 may be performed at least partially by visualization component 106.

FIG. 6 is a screen shot illustrating an example of a user interface display 300 which may be provided by user interface 112, according to some embodiments of the invention. Display 300 may include sub-displays 310, 420 and 430, and one or more other displays. Display 300 also may include a lengthwise location control 410 for a user to select a lengthwise location along a representation of a region from which pressure information was detected. This lengthwise location may define the lengthwise position within the region for which pressure information is being represented in the one or more sub-displays.

Sub-display 310 may include a three-dimensional mesh plot 306 which may be similar to or the same as the three-dimensional mesh plot 306 discussed above with respect to FIG. 3. Visual representation 310 may include a lengthwise location indicator 406, which may represent the lengthwise location represented by lengthwise control 410. Lengthwise location indicator 406 may indicate (on visual representation 310) the lengthwise position within the region for which pressure information detected at multiple circumferential positions is represented by one or more sub-displays (e.g., sub-displays 420 and/or 430).

Sub-display 420 may include a polar line trace plot 412. Polar line trace plot 412 may be a visual representation that represents pressure information detected by sensors positioned at lengthwise position 404 along the lengthwise dimension of a region. Polar line trace plot 412 may represent pressure information detected at a plurality of circumferential positions at the lengthwise position. For example, in FIG. 6, polar line trace plot 412 represents pressure information detected at positions that extend approximately 360° around the circumferential dimension.

Polar line trace plot 412 may include a line trace 416. Line trace 416 may represent pressure values of pressure information detected at a plurality of circumferential positions for the same lengthwise position. For example, the distance of line trace 416 from a center axis of the circumferential dimension at a circumferential location on polar line trace plot 412 may represent a pressure value detected at a corresponding circumferential position within the region. A greater distance from the center axis may represent a lower pressure and a lesser distance from the center axis may represent higher pressure, or vice-versa.

Labels 414 may be included in polar line trace plot 412. Labels 414 may indicate one or more locations (e.g., “Posterior”, “Anterior”, “Left”, Right”) on polar line trace plot 412 that correspond with positions with respect to an organism. In some embodiments, locations of these labels with respect to the detected pressure information may be determined based on an established reference position, for example, a reference position established by the position of probe 210.

Sub-display 430 may include a polar tone plot 418, according to some embodiments of the invention. Polar tone plot 418 may be a visual representation that represents pressure information detected by sensors positioned at different circumferential positions at lengthwise position 404. For example, in FIG. 6, polar tone plot 418 represents pressure information detected at positions that extend approximately 360° around the circumferential dimension.

Polar tone plot 418 may include a tone annulus 416. Tone annulus 416 may represent pressure values of pressure information detected at a plurality of circumferential positions. For example, each tone may represent a respective pressure range, for example, similar to as described above in relation to FIG. 4. The pressure range represented by a tone at a circumferential location on polar tone plot 418 may represent a pressure value (falling within the range) detected at a corresponding circumferential position within the region. Labels 414 may also be included in polar tone plot 418.

A sub-display may provide one or more visual representations. For example, a sub-display may provide both a polar line trace plot and a polar tone plot.

In act 706, a user-movable control (e.g., control 410 and/or indicator 406) may be provided that enables a user to select a lengthwise position within a region for which pressure information is to be visually represented. Act 706 may be performed at least partially by visualization component 106.

Lengthwise location control 410 may include a region representation 408 for representing positions along the lengthwise dimension of a region within which pressure information was detected. Lengthwise location control 410 also may include a lengthwise marker 402, which may be movable by a user to select a lengthwise position 404 along the lengthwise dimension of the region for which pressure information is to be represented by a sub-display (e.g., sub-displays 420 and/or 430). A location of lengthwise marker 402 with respect to region representation 408 may represent a respective position along the lengthwise dimension of the region.

In some embodiments, lengthwise location indicator 406 may be a control that is movable by a user to select a lengthwise position 404 along the lengthwise dimension of the region for which pressure information is to be represented by a sub-display (e.g., sub-displays 420 and/or 430).

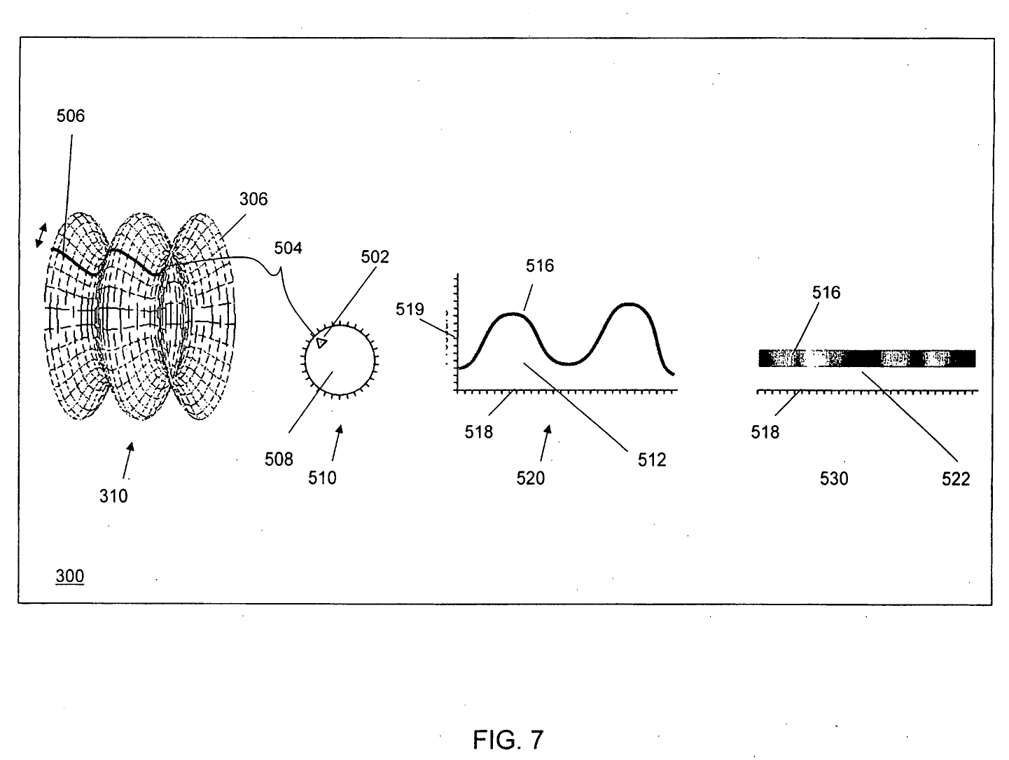

In act 708, a second visual representation may be provided which represents pressure information detected along a lengthwise dimension of a region at a circumferential position. In some embodiments, more than one visual representation may be provided, as will be described in more detail below. One or more visual representations illustrated in FIG. 7 may be provided in act 708, e.g., line trace plot 512 and tone plot 522. Act 708 may be performed at least partially by visualization component 106.

FIG. 7 is a screen shot illustrating an example of a user interface display 300, which may be provided by user interface 112, according to some embodiments of the invention. Display 300 may include one or more displays, including any of sub-displays 310, 520 and 530.

Sub-display 310 may include a three-dimensional mesh plot 306 which may be similar to or the same as the three-dimensional mesh plot 306 discussed above with respect to FIG. 3. Visual representation 310 may include a circumferential location indicator 506, which may represent the circumferential location represented by circumferential location control 510.

Sub-display 520 may include a lengthwise line trace plot 512. Lengthwise line trace plot 512 may be a visual representation that represents pressure information detected by sensors positioned at different lengthwise positions along the lengthwise dimension of a region at circumferential position 504.

Lengthwise line trace plot 512 may include a line trace 516. Line trace 516 may represent pressure values of pressure information detected at a plurality of lengthwise positions. For example, the distance of line trace 516 from a lengthwise axis 518 at a location along the lengthwise axis 518 may represent a pressure value detected at a corresponding lengthwise position within the region. A greater distance from the lengthwise axis 518 (as indicated by pressure axis 519) may represent a higher pressure and a lesser distance from the lengthwise axis 518 may represent a lower pressure.

Sub-display 530 may include a tone plot 522. Tone plot 522 may be a visual representation that represents pressure information detected by a plurality of sensors positioned along the lengthwise dimension of a region at circumferential position 504.

In act 710, a user-movable control may be provided that enables a user to select a circumferential position within a region for which pressure information is to be visually represented. The location of a marker within a visual representation may represent a circumferential position within the region. In some embodiments, the user-movable control may be similar to or the same as circumferential location control 510. Act 710 may be performed at least partially by visualization component 106.

Display 300 may also include a circumferential location control 510 for a user to select a circumferential location along a representation of a region from which pressure information was detected. This circumferential location may define the circumferential position within the region for which pressure information is being represented in the one or more sub-displays.

Circumferential location control 510 may include a region representation 508 for representing positions along the circumferential dimension of a region (e.g., a region at which probe 210 is/was positioned). Circumferential location control 510 also may include a circumferential marker 502, which may be movable by a user to select a circumferential position 504 along the circumferential dimension of the region for which pressure information is to be represented by a sub-display (e.g., sub-displays 520 and/or 530). A location of circumferential marker 502 with respect to region representation 508 may represent a respective position along the circumferential dimension of the region.

Circumferential location indicator 506 may indicate (on visual representation 310) the circumferential position within the region for which pressure information detected at multiple lengthwise positions is represented by one or more sub-displays (e.g., sub-displays 520 and/or 530). In some embodiments, circumferential location indicator 506 may be a control movable by a user to select a circumferential position 504 along the circumferential dimension of the region for which pressure information is to be represented by a sub-display (e.g., sub-displays 520 and/or 530).

Tone plot 522 may include a tone bar 516. Tone bar 516 may represent pressure values of pressure information detected at a plurality of lengthwise positions. Each tone may represent a different pressure range, for example, at least similar to as described above in relation to FIG. 4. The pressure range represented by a tone at a lengthwise location on tone plot 522 may represent a pressure value (falling within the range) detected at a corresponding lengthwise position within the region.

Method 700 may include fewer acts than those described above. Method 700 may include additional acts. Further, some or all of the acts may be performed concurrently to other acts, and need not necessarily be performed in the order described above.

A visual representation may represent one or more average pressure values, maximum pressure values, and/or minimum pressure values of pressure values detected along a dimension of a region.

For example, a visual representation (e.g., polar line trace plot 412 and/or polar tone plot 418) may represent average pressure values along the lengthwise dimension of the region, for multiple circumferential positions. For a given circumferential position, an average lengthwise pressure value may be calculated by averaging the pressure values along the lengthwise dimension of the region at the circumferential position. The average lengthwise pressure values for multiple circumferential positions may be represented by, for example, a polar line trace and/or tone annulus. A visual representation of the maximum pressure values and/or minimum pressure values may be provided in a similar manner, calculating minimum pressure values and/or maximum pressure values instead of average pressure values.

As another example, a visual representation (e.g., lengthwise line trace plot 512 and/or tone plot 418) may represent average pressure values along the circumferential dimension of the region, for multiple lengthwise positions. For a given lengthwise position, an average circumferential pressure value may be calculated by averaging the pressure values along the circumferential dimension of the region at the lengthwise position. The average circumferential pressure values for multiple lengthwise positions may be represented by, for example, a lengthwise line trace plot and/or a tone plot. A visual representation of the maximum pressure values and/or minimum pressure values may be provided in a similar manner, calculating minimum pressure values and/or maximum pressure values instead of average pressure values.

A visual representation may represent one or more average pressure values, maximum pressure values, and/or minimum pressure values of pressure values detected during a particular time period. Such average pressure value may be provided by performing calculations (e.g., averaging, finding maxima, or finding minima) on pressure values detected during the time period. Such average pressure values may be represented on any suitable visual representation discussed above.

A visual representation may represent pressure information detected over a period of time. For example, the visual representation may be altered in time to represent pressure information detected at various points during the period. In some embodiments, a visual representation may represent pressure information in real time and may be updated at regular intervals. In some embodiments, a visual representation may represent pressure information detected over a period of time post hoc, i.e., after the detection has occurred. In such a post-hoc embodiment, the visual representation may play back the detected pressure information at a same rate at which it was detected, or the pressure information may be played back at a different rate (faster or slower) and/or paused at various times.

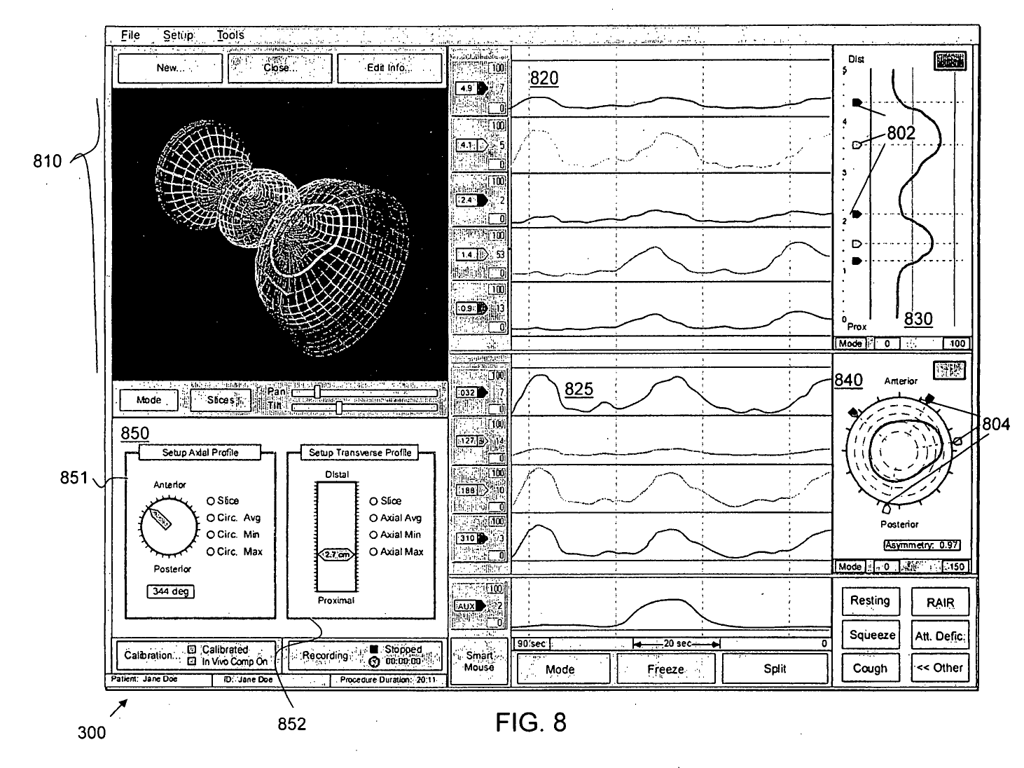

FIG. 8 is a screen shot illustrating an example of a user interface display 300 which may be provided by user interface 112, according to some embodiments of the invention. User interface display 300 may include five sub-displays 810, 820, 825, 830, and 840, which may each include at least one visual representation.

Sub-display 850 may include one or more controls for a user to select a position within a region (within which pressure information was detected, e.g., by probe 210) for which pressure information is to be represented in sub-displays 820, 825, 830, and 840. The controls included in sub-display 850 may be similar to circumferential location control 510 and/or lengthwise location control 410 discussed above.

Control 851, for example, may enable a user to select the circumferential position for which pressure information along the lengthwise dimension is to be displayed in sub-displays 820 and/or 830. Control 851 may enable a user to select from one of the following: a circumferential position; an average pressure value for all circumferential positions; a minimum pressure value for all circumferential positions or a maximum pressure value for all circumferential positions.

Control 851 may control the information displayed in sub-displays 820 and 830. Sub-display 830 may include a lengthwise plot which may be similar to lengthwise line trace plot 512 discussed above. Sub-display 830 may represent pressure information detected at a point in time for a plurality of positions along a lengthwise dimension of a region, at the circumferential position determined by control 851. Markers 802 may be provided in sub-display 830 which each indicate a lengthwise position within the region for which pressure information detected over time is represented in sub-display 820. In some embodiments, markers 802 may be individually movable to enable a user to select a lengthwise position to be represented in sub-display 820.

Sub-display 820 may include a temporal representation (e.g., a line trace plot and/or a contour plot) which may represent pressure information detected over time (e.g., in real-time or post-hoc). Sub-display 820 may represent pressure information detected over time at the circumferential position determined by control 851 and the lengthwise positions indicated by markers 802. In some embodiments, a temporal representation may move in time along the temporal dimension of the representation to represent a change in the pressure information over time. For example, one or more line traces displayed in sub-display 820 may move from right to left at time progresses.

Control 852, for example, may enable a user to select the lengthwise position for which pressure information along the circumferential dimension is to be displayed in sub-displays 825 and/or 840. Control 852 may enable a user to select from one of the following: a lengthwise position; an average pressure value for all lengthwise positions; a minimum pressure value for all lengthwise positions or a maximum pressure value for all lengthwise positions.

Control 852 may control the information displayed in sub-displays 825 and 840. Sub-display 840 may include a polar plot which may be similar to polar line trace plot 412 discussed above. Sub-display 840 may represent pressure information detected at a point in time for a plurality of positions along a circumferential dimension of a region, at the lengthwise position determined by control 852. Markers 804 may be provided in sub-display 840 which each indicate a circumferential position within the region for which pressure information detected over time is represented in sub-display 825. In some embodiments, markers 802 may be individually movable to enable a user to select a circumferential position within to be represented in sub-display 825.

Sub-display 825 may include a temporal representation (e.g., a line trace plot and/or a contour plot) which may represent pressure information detected over time (e.g., in real-time or post-hoc). Sub-display 825 may represent pressure information detected over time at the lengthwise position determined by control 852 and the circumferential positions indicated by markers 804. In some embodiments, a temporal representation may move in time along the temporal dimension of the representation to represent a change in the pressure information over time. For example, one or more line traces displayed in sub-display 825 may move from right to left at time progresses.

Sub-display 810 may include a multidimensional plot which may be similar to three-dimensional mesh plot 306 discussed above.

Sub-displays 820 and/or 825 may include a contour plot representing pressure information detected over time. As used herein, a “contour plot” is a visual representation that visually indicates values detected over time at locations on a temporal plot having a temporal dimension corresponding to time and a spatial dimension corresponding to a region. A contour plot may represent to a user pressure data derived from pressure information measured by sensors at a plurality of positions over time. A contour plot may include one or more tones which each represent a pressure range.

Methods described herein, acts thereof and various embodiments and variations of this method and these acts, individually or in combination, may be defined by computer-readable signals tangibly embodied on or more computer-readable media, for example, non-volatile recording media, integrated circuit memory elements, or a combination thereof. Computer readable media can be any available media that can be accessed by a computer. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, other types of volatile and non-volatile memory, any other medium which can be used to store the desired information and which can accessed by a computer, and any suitable combination of the foregoing.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, wireless media such as acoustic, RF, infrared and other wireless media, other types of communication media, and any suitable combination of the foregoing.

Computer-readable signals embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, or C++, Fortran, Pascal, Eiffel, Basic, COBOL, etc., or any of a variety of combinations thereof. The computer-readable media on which such instructions are embodied may reside on one or more of the components of any of systems described herein, may be distributed across one or more of such components, and may be in transition therebetween.

The computer-readable media may be transportable such that the instructions stored thereon can be loaded onto any computer system resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a processor to implement the above-discussed aspects of the present invention.

It should be appreciated that any single component or collection of multiple components of a computer system, for example, the computer system described in relation to FIGS. 9 and 10 that perform the functions described herein can be generically considered as one or more controllers that control such functions. The one or more controllers can be implemented in numerous ways, such as with dedicated hardware and/or firmware, using a processor that is programmed using microcode or software to perform the functions recited above or any suitable combination of the foregoing.

Various embodiments according to the invention may be implemented on one or more computer systems. These computer systems, may be, for example, general-purpose computers such as those based on Intel PENTIUM-type processor, Motorola PowerPC, Sun UltraSPARC, Hewlett-Packard PA-RISC processors, or any other type of processor. It should be appreciated that one or more of any type computer system may be used to convert text to speech and/or edit speech on a portable audio device according to various embodiments of the invention. Further, the software design system may be located on a single computer or may be distributed among a plurality of computers attached by a communications network.

A general-purpose computer system according to one embodiment of the invention is configured to perform convert text to speech and/or edit speech on a portable audio device. It should be appreciated that the system may perform other functions and the invention is not limited to having any particular function or set of functions.

For example, various aspects of the invention may be implemented as specialized software executing in a general-purpose computer system 1700 such as that shown in FIG. 9. The computer system 1700 may include a processor 1703 connected to one or more memory devices 1704, such as a disk drive, memory, or other device for storing data. Memory 1704 is typically used for storing programs and data during operation of the computer system 1700. Components of computer system 1700 may be coupled by an interconnection mechanism 1705, which may include one or more busses (e.g., between components that are integrated within a same machine) and/or a network (e.g., between components that reside on separate discrete machines). The interconnection mechanism 1705 enables communications (e.g., data, instructions) to be exchanged between system components of system 1700. Computer system 1700 also includes one or more input devices 1702, for example, a keyboard, mouse, trackball, microphone, touch screen, and one or more output devices 1701, for example, a printing device, display screen, speaker. In addition, computer system 1700 may contain one or more interfaces (not shown) that connect computer system 1700 to a communication network (in addition or as an alternative to the interconnection mechanism 1705.

The storage system 1706, shown in greater detail in FIG. 10, typically includes a computer readable and writeable nonvolatile recording medium 1801 in which signals are stored that define a program to be executed by the processor or information stored on or in the medium 1801 to be processed by the program. The medium may, for example, be a disk or flash memory. Typically, in operation, the processor causes data to be read from the nonvolatile recording medium 1801 into another memory 1802 that allows for faster access to the information by the processor than does the medium 1801. This memory 1802 is typically a volatile, random access memory such as a dynamic random access memory (DRAM) or static memory (SRAM). It may be located in storage system 1706, as shown, or in memory system 1704, not shown. The processor 1703 generally manipulates the data within the integrated circuit memory 1704, 1802 and then copies the data to the medium 1801 after processing is completed. A variety of mechanisms are known for managing data movement between the medium 1801 and the integrated circuit memory element 1704, 1802, and the invention is not limited thereto. The invention is not limited to a particular memory system 1704 or storage system 1706.

The computer system may include specially-programmed, special-purpose hardware, for example, an application-specific integrated circuit (ASIC). Aspects of the invention may be implemented in software, hardware or firmware, or any combination thereof. Further, such methods, acts, systems, system elements and components thereof may be implemented as part of the computer system described above or as an independent component.

Although computer system 1700 is shown by way of example as one type of computer system upon which various aspects of the invention may be practiced, it should be appreciated that aspects of the invention are not limited to being implemented on the computer system as shown in FIG. 9. Various aspects of the invention may be practiced on one or more computers having a different architecture or components that that shown in FIG. 9.

Computer system 1700 may be a general-purpose computer system that is programmable using a high-level computer programming language. Computer system 1700 may be also implemented using specially programmed, special purpose hardware. In computer system 1700, processor 1703 is typically a commercially available processor such as the well-known Pentium class processor available from the Intel Corporation. Many other processors are available. Such a processor usually executes an operating system which may be, for example, the Windows® 95, Windows® 98, Windows NT®, Windows® 2000 (Windows® ME) or Windows® XP operating systems available from Microsoft Corporation, MAC OS System X available from Apple Computer, the Solaris Operating System available from Sun Microsystems, UNIX available from various sources or Linux available from various sources. Many other operating systems may be used.

The processor and operating system together define a computer platform for which application programs in high-level programming languages are written. It should be understood that the invention is not limited to a particular computer system platform, processor, operating system, or network. Also, it should be apparent to those skilled in the art that the present invention is not limited to a specific programming language or computer system. Further, it should be appreciated that other appropriate programming languages and other appropriate computer systems could also be used.

One or more portions of the computer system may be distributed across one or more computer systems (not shown) coupled to a communications network. These computer systems also may be general-purpose computer systems. For example, various aspects of the invention may be distributed among one or more computer systems configured to provide a service (e.g., servers) to one or more client computers, or to perform an overall task as part of a distributed system. For example, various aspects of the invention may be performed on a client-server system that includes components distributed among one or more server systems that perform various functions according to various embodiments of the invention. These components may be executable, intermediate (e.g., IL) or interpreted (e.g., Java) code which communicate over a communication network (e.g., the Internet) using a communication protocol (e.g., TCP/IP).

It should be appreciated that the invention is not limited to executing on any particular system or group of systems. Also, it should be appreciated that the invention is not limited to any particular distributed architecture, network, or communication protocol.

Various embodiments of the present invention may be programmed using an object-oriented programming language, such as SmallTalk, Java, C++, Ada, J# (J-Sharp) or C# (C-Sharp). Other object-oriented programming languages may also be used. Alternatively, functional, scripting, and/or logical programming languages may be used. Various aspects of the invention may be implemented in a non-programmed environment (e.g., documents created in HTML, XML or other format that, when viewed in a window of a browser program, render aspects of a graphical-user interface (GUI) or perform other functions). Various aspects of the invention may be implemented as programmed or non-programmed elements, or any combination thereof.

Having now described some illustrative embodiments of the invention, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments. Further, for the one or more means-plus-function limitations recited in the following claims, the means are not intended to be limited to the means disclosed herein for performing the recited function, but are intended to cover in scope any equivalent means, known now or later developed, for performing the recited function.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1.-20. (canceled)

21. A method of visually representing pressure information detected by a plurality of sensors disposed at positions within a region of an organism, the region having a lengthwise dimension and a circumferential dimension, the circumferential dimension having a center axis, the method comprising acts of:
(A) receiving pressure data representing the pressure information; and
(B) providing, at least partially based on the pressure data, a first visual representation of the pressure information illustrating at least a portion of the region, the portion spanning at least two positions along the lengthwise dimension and subtending, at each of the at least two positions along the lengthwise dimension, an angle greater than 180 degrees with respect to the center axis.

22. The method of claim 21, wherein the angle is greater than 270 degrees.

23. The method of claim 22, wherein the angle is approximately 360 degrees.

24. The method of claim 21, wherein the act (B) comprises providing a polar plot illustrating a circumferential pressure distribution representing pressure values detected within the portion of the region.

25. The method of claim 24, wherein the polar plot comprises a line trace.

26. The method of claim 24, wherein the polar plot comprises a tone-encoded plot.

27. The method of claim 24, wherein the first visual representation is displayed on a user interface display, and the method further comprises an act of:
(C) providing a user-movable control on the user interface display, the control enabling a user to select a position along the lengthwise dimension of the probe for which the first visual representation is to be provided in the act (B).

28. The method of claim 21, wherein the first visual representation is a multi-dimensional representation representing the pressure information detected at a plurality of positions along the lengthwise dimension and circumferential dimension of the region.

29. The method of claim 21, wherein the first visual representation is a two-dimensional plot representing the pressure information at a plurality of positions along the lengthwise dimension and circumferential dimension of the region.

30. The method of claim 29, wherein the first visual representation is a contour plot.

31. The method of claim 28, wherein the first visual representation comprises a three-dimensional mesh plot comprising a mesh.

32. The method of claim 31, wherein the pressure information is represented at least partially by a radial location of at least a portion of the mesh, the radial location being along a radial dimension of the mesh plot, the radial dimension representing a dimension of the region of the organism that is substantially orthogonal to the lengthwise dimension.

33. The method of claim 31, wherein the pressure information is represented at least partially by a tone-encoding of the three-dimensional mesh plot.

34. The method of claim 21, further comprising an act of:
(C) providing a second visual representation representing the pressure information detected at a plurality of positions along the lengthwise dimension of the region, each position of the plurality of positions having substantially a same circumferential position along the circumferential dimension.

35. The method of claim 34, wherein the first and second visual representations are displayed concurrently.

36. The method of claim 34, wherein the second visual representation comprises a line trace.

37. The method of claim 34, wherein the second visual representation comprises a tone-encoded plot.

38. The method of claim 34, wherein the first visual representation is displayed on a user interface display, and the method further comprises an act of:
(D) providing a user-movable control on the user interface display, the control enabling a user to select a circumferential position along the circumferential dimension of the probe for which the second visual representation is to be provided in the act (C).

39. The method of claim 34, wherein the pressure information was detected by the plurality of sensors disposed at the positions within the region over a period of time such that the pressure information represents the pressure information detected over the period of time, the method further comprising an act of:
(D) altering the second visual representation to represent a change in the pressure information over the period of time.

40. The method of claim 21, wherein the second visual representation represents an average pressure value, maximum pressure value, and/or minimum pressure value of pressure values detected during a particular time period.

41. The method of claim 21, wherein the second visual representation represents, for a plurality of lengthwise positions along the lengthwise dimension of the region, average pressure values, maximum pressure values and/or minimum pressure values determined from pressure values detected at a plurality of positions along the circumferential dimension of the region at the each of the plurality of lengthwise positions.

42. The method of claim 21, further comprising acts of:
(C) receiving updated pressure data representing the pressure information; and
(D) providing, at least partially based on the updated pressure data, a second visual representation of the pressure information illustrating at least a portion of the region, the portion subtending, with respect to the center axis, an angle greater than 180 degrees.

43. The method of claim 42, wherein the act B further comprises displaying the first visual representation on a human perceptible medium and the act D further comprises displaying the second visual representation on the human perceptible medium, wherein the second visual representation is displayed soon enough in time after the first visual representation such that the first and second visual representations appear on the human perceptible medium as a single, moving temporal representation.

44. The method of claim 21, wherein the first visual representation represents an average pressure value, maximum pressure value, and/or minimum pressure value of pressure values detected during a particular time period.

45. The method of claim 21, wherein the first visual representation represents, for a plurality of circumferential positions along the circumferential dimension of the region, average pressure values, maximum pressure values and/or minimum pressure values determined from pressure values detected at a plurality of positions along the lengthwise dimension of the region at the each of the plurality of circumferential positions.

46. The method of claim 21, further comprising an act of:
(C) generating interpolated pressure values at least partially based on the pressure information, the interpolated pressure values representing pressure information for positions between the positions of the plurality of sensors.

47. A computer program product comprising:
a computer-readable medium; and
computer-readable signals, stored on the computer-readable medium, that define instructions that, as a result of being executed by a computer, control the computer to perform a method of visually representing pressure information detected by a plurality of sensors disposed at positions within a region of an organism, the region having a lengthwise dimension and a circumferential dimension, the circumferential dimension having a center axis, the method comprising acts of:
(A) receiving pressure data representing the pressure information; and
(B) providing, at least partially based on the pressure data, a first visual representation of the pressure information illustrating at least a portion of the region, the portion spanning at least two positions along the lengthwise dimension and subtending, at each of the at least two positions along the lengthwise dimension, an angle greater than 180 degrees with respect to the center axis.

48. A system for visually representing pressure information detected by a plurality of sensors disposed at positions within a region of an organism, the region having a lengthwise dimension and a circumferential dimension, the circumferential dimension having a center axis, the system comprising:
a visualization component operative to:
receive pressure data representing the pressure information; and
provide, at least partially based on the pressure data, a first visual representation of the pressure information illustrating at least a portion of the region, the portion spanning at least two positions along the lengthwise dimension and subtending, at each of the at least two positions along the lengthwise dimension, an angle greater than 180 degrees with respect to the center axis.
